SEQUENCE LISTING (33 sequences)

SEQ ID NO: 1 - DNA, 24 bp, Artificial Sequence
  Description of Artificial Sequence PTEN forward primer
atgacaatcatgttgcagcaattc

SEQ ID NO: 2 - DNA, 25 bp, Artificial Sequence
  Description of Artificial Sequence PTEN reverse primer
cgatgcaataaatatgcacaaatca

SEQ ID NO: 3 - DNA, 26 bp, Artificial Sequence
  Description of Artificial Sequence PTEN probe
gtaaagctggaaagggacggactggt

SEQ ID NO: 4 - DNA, 21 bp, Artificial Sequence
  Description of Artificial Sequence fas forward primer
tccaagacacagctgagcaga

SEQ ID NO: 5 - DNA, 22 bp, Artificial Sequence
  Description of Artificial Sequence fas reverse primer
tgcatcactcttcccatgagat

SEQ ID NO: 6 - DNA, 26 bp, Artificial Sequence
  Description of Artificial Sequence fas probe
agtccagctgctcctgtgctggtacc

SEQ ID NO: 7 - DNA, 19 bp, Artificial Sequence
  Description of Artificial Sequence apolipoprotein b forward primer
cgtgggctccagcattcta

SEQ ID NO: 8 - DNA, 21 bp, Artificial Sequence
  Description of Artificial Sequence apolipoprotein b reverse primer
agtcatttctgcctttgcgtc

SEQ ID NO: 9 - DNA, 22 bp, Artificial Sequence
  Description of Artificial Sequence apolipoprotein b probe
ccaatggtcgggcactgctcaa

SEQ ID NO: 10 - DNA, 21 bp, Artificial Sequence
  Description of Artificial Sequence microsomal triglyceride transfer protein forward primer
gagcggtctggatttacaacg

SEQ ID NO: 11 - DNA, 24 bp, Artificial Sequence
  Description of Artificial Sequence microsomal triglyceride transfer protein reverse primer
aggtagtgacagatgtggcttttg

SEQ ID NO: 12 - DNA, 23 bp, Artificial Sequence
  Description of Artificial Sequence microsomal triglyceride transfer protein probe
caaaccaggtgctgggcgtcagt

SEQ ID NO: 13 - DNA, 15 bp, Artificial Sequence
  Description of Artificial Sequence murine cyclophilin a forward primer
tcgccgcttgctgca

SEQ ID NO: 14 - DNA, 17 bp, Artificial Sequence
  Description of Artificial Sequence murine cyclophilin a reverse primer
atcggccgtgatgtcga

SEQ ID NO: 15 - DNA, 23 bp, Artificial Sequence
  Description of Artificial Sequence murine cyclophilin a probe
ccatggtcaaccccaccgtgttc

SEQ ID NO: 16 - DNA, 20 bp, Artificial Sequence
  Description of Artificial Sequence PTEN antisense
ctgctagcctctggatttga

SEQ ID NO: 17 - DNA, 20 bp, Artificial Sequence
  Description of Artificial Sequence PTEN antisense
cttctggcatccggtttaga

SEQ ID NO: 18 - DNA, 20 bp, Artificial Sequence
  Description of Artificial Sequence PTEN antisense
ctgctagcctctggatttga

SEQ ID NO: 19 - DNA, 20 bp, Artificial Sequence
  Description of Artificial Sequence PTEN antisense
cttctggcatccggtttaga

SEQ ID NO: 20 - DNA, 20 bp, Artificial Sequence
  Description of Artificial Sequence Fas antisense
tccagcactttcttttccgg

SEQ ID NO: 21 - DNA, 20 bp, Artificial Sequence
  Description of Artificial Sequence Fas antisense
tccatctccttttatgccgg

SEQ ID NO: 22 - DNA, 20 bp, Artificial Sequence
  Description of Artificial Sequence MTTP antisense
cccagcacctggtttgccgt

SEQ ID NO: 23 - DNA, 20 bp, Artificial Sequence
  Description of Artificial Sequence Apo B antisense
gtccctgaagatgtcaatgc

SEQ ID NO: 24 - DNA, 21 bp, Artificial Sequence
  Description of Artificial Sequence PTEN antisense containing uridine nucleotides
cugcuagccucuggauuugtt

SEQ ID NO: 25 - DNA, 21 bp, Artificial Sequence
  Description of Artificial Sequence PTEN sense containing uridine nucleotides
caaauccagaggcuagcagtt

SEQ ID NO: 26 - DNA, 21 bp, Artificial Sequence
  Description of Artificial Sequence PTEN antisense containing uridine nucleotides
cuucuggcauccgguuuagtt

SEQ ID NO: 27 - DNA, 21 bp, Artificial Sequence
  Description of Artificial Sequence PTEN sense containing uridine nucleotides
cuaaaccggaugccagaagtt

SEQ ID NO: 28 - RNA, 21 nt, Artificial Sequence
  Description of Artificial Sequence PTEN antisense
cugcuagccucuggauuugac

SEQ ID NO: 29 - RNA, 21 nt, Artificial Sequence
  Description of Artificial Sequence PTEN sense
gucaaauccagaggcuagcag

SEQ ID NO: 30 - DNA, 21 bp, Artificial Sequence
  Description of Artificial Sequence Fas antisense containing uridine nucleotides
gucugguuugcacuugcactt

SEQ ID NO: 31 - DNA, 21 bp, Artificial Sequence
  Description of Artificial Sequence Fas sense containing uridine nucelotides
gugcaagugcaaaccagactt

SEQ ID NO: 32 - DNA, 21 bp, Artificial Sequence
  Description of Artificial Sequence Fas antisense containing uridine nucleotides
gugucguguucaguuccactt

SEQ ID NO: 33 - DNA, 21 bp, Artificial Sequence
  Description of Artificial Sequence Fas sense containing uridine nucleotides
guggaacugaacacgacactt

(19) United States
(12) Patent Application Publication
(10) Pub. No.: US 2005/0164209 A1
(43) Pub. Date: Jul. 28, 2005

(54) Hepatocyte free uptake assays

(76) Inventors: C. Frank Bennett, Carlsbad, CA (US); Robert McKay, Poway, CA (US); Brett P. Monia, Encinitas, CA (US); Brenda F. Baker, Carlsbad, CA (US)

Correspondence Address: COZEN O'CONNOR, P.C., 1900 MARKET STREET, PHILADELPHIA, PA, 19103-3508, US

(21) Appl. No.: 10/763,367

(22) Filed: Jan. 23, 2004

(51) Int. Cl. C12Q 1/68; C12P 19/34; C12N 5/08

(52) U.S. Cl. 435/6; 435/91.1; 435/366

(57) ABSTRACT

The present invention provides methods of identifying an oligomeric compound having bioactivity in vivo, methods of identifying a small interfering RNA having bioactivity in vivo, and kits.

### FIELD OF THE INVENTION

The present invention is directed, in part, to methods of identifying an oligomeric compound having bioactivity in vivo and to kits therefore.

### BACKGROUND OF THE INVENTION

The concept of using antisense oligonucleotides (ASOs) to reduce protein expression was first proposed by Zamecnik and Stephenson in 1978 when they demonstrated that an oligonucleotide complementary to 13 nucleotides of the Rous sarcoma virus 35S RNA inhibited virus production in Rous infected chick embryo fibroblasts (Zamecnik et al., Proc. Natl. Acad. Sci., 1978, 75, 280-284). Advances in antisense therapeutics since this time have been substantial, with the first therapeutic ASO being approved for human use in 1998 (Marwick, J. Am. Med. Assoc., 1998, 280, 871). The recent introduction of RNA interference as a method to analyze gene function in invertibrates and plants (Fraser et al., Nature, 2000, 408, 325-330) has suggested that double-stranded RNA, specifically small nucleotide interfering RNAs (siRNAs), may also have therapeutic applications (Vickers et al., J. Biol. Chem., 2003, 278, 7108-7118).

When double-stranded RNA molecules are introduced into cells they are metabolized to small 21-23 nucleotide siRNAs with two-nucleotide (2-nt) 3′-overhangs via the endogenous ribonuclease Dicer (Grishok et al., 2000, Science, 287, 2494-2497; and Zamore et al., 2000, Cell, 101, 25-33). Inside cells, siRNA molecules bind to an RNA-induced silencing protein complex. This complex, which possesses helicase activity, unwinds the double-stranded siRNA, thereby allowing the antisense strand to bind to the targeted RNA. An endonuclease then hydrolyzes the target RNA (Zamore et al., 2000, Cell, 101, 25-33; and Zamore, 2002, Science, 296, 1265-1269). Since ultimately a single stranded RNA molecule binds to the target RNA molecule according to Watson-Crick base pairing rules, siRNA driven RNA interference is essentially an antisense mechanism of action (Vickers et al., J. Biol. Chem., 2003, 278, 7108-7118). siRNA duplexes used for silencing mammalian genes in cultured cells are usually chemically synthesized 21-23 nucleotide (21-23-nt) siRNAs, where the siRNA's sense and antisense strands are paired, containing 2-nt 3′-overhangs (Harborth et al., J. Cell. Sci., 2001, 114, 4557-4565). siRNA molecules were designed with a 2 nucleotide (2 nt) 3′-overhang because this form of siRNA has been shown to be most effective in vitro (Elbashir et al., Nature, 2001, 411, 494-498). The 5′-hydroxyl is not blocked by methylation or a 5′-phosphodiester linkage, as both prevent the 5′-phosphorylation of the antisense siRNA, a step necessary for target RNA degradation inside cells (Nykanen et al., Cell, 2001, 107, 309-321; and Schwartz et al., Mol. Cell., 2002, 10, 537-548).

Zamore and others have reported that single-stranded antisense oligonucleotides are less potent and less effective than siRNAs at reducing gene transcript levels (Zamore et al., 2000, Cell, 101, 25-33; and Caplen et al., Proc. Natl. Acad. Sci. USA, 2001, 98, 9742-9747). As the antisense molecules used in those studies were single-stranded unmodified RNA, which are rapidly degraded by endogenous nucleases, here we compare antisense siRNA molecules to ‘second generation’ phosphorothioate (PS) oligodeoxynucleotides modified to contain 2′-O-methoxyethyls (MOEs), both in vitro and in vivo. These second generation antisense oligonucleotides are chimeric molecules, which by design, contain a stretch of RNAse H sensitive 2′deoxy residues in the middle, flanked on both sides with a region of 2′MOE modifications. These molecules, termed MOE gapmers, take advantage of: 1) 2′-MOE modifications, which form higher affinity complexes and possess higher nuclease resistance relative to ‘first generation’ PS oligonucleotides, resulting in increased ASO potency both in vitro and in vivo (Altmann et al., Biochem. Soc. Trans., 1996, 24, 630-637; Dean, N. M., Pharmacology of 2′-O-(2-methoxy)-ethyl modified antisense oligonucleotides, in Antisense Technology: Principles, Strategies and Applications, S. Crooke, Editor, Marcel Dekker, 2001; and Kurreck, Eur. J. Biochem., 2003, 270, 1628-1644); and 2) PS 2′deoxyoligonucleotides, which when duplexed with RNA, serve as efficient substrates for the robust endogenous RNAse H antisense-mediated cleavage of RNA (Baker et al., Biochim. Biophys. Acta, 1999, 1489, 3-18). Indeed, antisense MOE gapmer reduction of target mRNA levels can be in the order of 85-90% of control levels (Crooke et al., Annu. Rev. Pharmacol. Toxicol., 1996, 36, 107-129; and Baker et al., Biochim. Biophys. Acta, 1999, 1489, 3-18).

Antisense oligonucleotides are known to preferentially accumulate in hepatic tissue in vivo (Cossum et al., J. Pharmacol. Exp. Ther., 1993, 267, 1181-1190; and Graham et al., J. Pharmacol. Exp. Therap., 1998, 286, 447-458). Nestle and colleagues have previously reported that cultured hepatocytes rapidly internalize antisense compounds in the absence of cationic lipid transfection reagents (Nestle et al., J. Invest. Dermatol., 1994, 103, 569-575). These observations are likely related to the remarkable transport rates displayed by hepatocytes, where fluid-phase endocytosis at the basolateral membrane is estimated to be 8% of the total membrane surface area per minute per cell (Crawford, Semin. Liver Dis., 1996, 16, 169-189).

The present invention was undertaken to provide a primary hepatocyte cell model that would demonstrate in vitro antisense oligonucleotide uptake and intracellular trafficking similar to postulated in vivo antisense oligonucleotide uptake and trafficking. In particular, the present invention demonstrates antisense oligonucleotide mediated target mRNA reduction in primary hepatocytes without cationic lipid carriers, analogous to that postulated to occur in vivo. The results described herein suggest that the mechanism of cellular uptake of single strand MOE gapmers and double strand siRNA are different. Single strand MOE gapmers, but likely not double strand siRNA, are taken up in hepatocytes in vivo and in vitro without aid of cationic lipids. When siRNA molecules are transfected into cells, they produce a dose dependent reduction of target gene expression.

### SUMMARY OF THE INVENTION

The present invention provides methods of identifying an oligomeric compound having bioactivity in vivo. A bioindicative cell is contacted with one or more candidate oligomeric compounds in vitro in the absence of a transfection reagent. The bioindicative cell is examined to determine whether it has an altered phenotype. If the bioindicative cell has an altered phenotype, one or more of the candidate oligomeric compounds comprises in vivo bioactivity. The oligomeric compound can be single stranded or double stranded. The oligomeric compound can be an oligonucleotide, peptide nucleic acid, small interfering RNA, micro RNA, micro RNA mimic, or any combination thereof. The bioindicative cell can be a mammalian tissue-derived cell such as a primary hepatocyte, primary keratinocyte, primary macrophage, primary fibroblast, primary pancreatic cell, or a stem cell. The altered phenotype can be an increase in uptake of the candidate oligomeric compound, decrease in expression of the mRNA produced from the gene to which the candidate oligomeric compound is targeted, or decrease in expression of the protein encoded by the gene or mRNA to which the candidate oligomeric compound is targeted.

In some embodiments, the candidate oligomeric compound can be designed to inhibit gene expression by hybridizing to a target through an antisense mechanism, such as an RNAse H-mediated inhibition of the target of the candidate oligomeric compound, an RNA interference-mediated inhibition of the target of the candidate oligomeric compound, splicing. In other embodiments, the candidate oligomeric compound is designed to inhibit RNA metabolism, transport, or protein metabolism by hybridizing to a target through an antisense mechanism.

The present invention also provides kits comprising an assay platform, a bioindicative cell, and a bioactive oligomeric compound.

The present invention also provides methods of identifying an oligomeric compound having bioactivity in vivo in which a primary hepatocyte is contacted with a candidate oligomeric compound in vitro in the absence of a transfection reagent. The primary hepatocyte is examined to determine whether it has a decreased level of an RNA to which the candidate oligomeric compound is targeted. If the primary hepatocyte has a decreased level of the RNA, then the candidate oligomeric compound comprises in vivo bioactivity.

The present invention also provides methods of identifying a small interfering RNA having bioactivity in vivo. A primary hepatocyte is contacted with a candidate small interfering RNA in vitro in the absence of a transfection reagent. The primary hepatocyte is examined to determine whether it has a decreased level of an RNA to which the candidate small interfering RNA is targeted. If the primary hepatocyte has a decreased level of the RNA, then the candidate small interfering RNA comprises in vivo bioactivity.

### DESCRIPTION OF EMBODIMENTS

The present invention provides methods of identifying an oligomeric compound having bioactivity in vivo, methods of identifying a small interfering RNA having bioactivity in vivo, and kits.

In particular, the present invention provides a system in which the uptake and activity of siRNA, in primary mouse hepatocytes, is compared to that observed, and well documented, for the 2′-MOE modified oligonucleotide RNase HI-mediated mRNA target reduction. A method is established looking at cellular uptake and distribution by using free, unassisted, RNA or DNA uptake, in which activity (gene silencing) is measured by RT-PCR, by using a lipid reagent as a carrier. The data shown herein demonstrates the loss of activity with siRNA in the absence of lipid-mediation treatments and the data is further supported with images of fluorescently labeled siRNA and its distribution in these cells. Furthermore, the data indicates that the loss of activity observed in this assay, which correlates with the lack of activity observed in vivo when mice are treated by conventional methods.

In the context of this invention, the term “oligomeric compound” refers to a plurality of naturally-occurring and/or non-naturally-occurring monomeric units joined together in a specific sequence. This term includes oligonucleotides, oligonucleosides, oligonucleotide analogs, oligonucleotide mimetics, and combinations of these. Oligomeric compounds are typically structurally distinguishable from, yet functionally inter-change-able with, naturally-occurring or synthetic wild-type oligonucleotides. Thus, oligomeric compounds include all such structures that function effectively to mimic the structure and/or function of a desired RNA or DNA strand, for example, by hybridizing to a target.

Oligomeric compounds are routinely prepared linearly but can be joined or otherwise prepared to be circular and may also include branching. Oligomeric compounds can included double stranded constructs such as for example two strands hybridized to form double stranded compounds. The double stranded compounds can be linked or separate and can include overhangs on the ends. In general an oligomeric compound comprises a backbone of linked monomeric subunits where each linked monomeric subunit is directly or indirectly attached to a heterocyclic base moiety. Oligomeric compounds may also include monomeric subunits that are not linked to a heterocyclic base moiety thereby providing a basic sites. The linkages joining the monomeric subunits, the sugar moieties or surrogates and the heterocyclic base moieties can be independently modified giving rise to a plurality of motifs for the resulting oligomeric compounds including hemimers, gapmers and chimeras.

In the context of this invention, the term “oligonucleotide” refers to an oligomer or polymer of ribonucleic acid (RNA) or deoxyribonucleic acid (DNA) or mimetics thereof. This term includes oligonucleotides composed of naturally-occurring nucleobases, sugars and covalent internucleoside (backbone) linkages as well as oligonucleotides having non-naturally-occurring portions that function similarly. Such modified or substituted oligonucleotides are often suitable over native forms because of desirable properties such as, for example, enhanced cellular uptake, enhanced affinity for nucleic acid target, and increased stability in the presence of nucleases.

Included in suitable oligomeric compounds are oligonucleotides such as antisense oligonucleotides, ribozymes, external guide sequence (EGS) oligonucleotides, alternate splicers, primers, probes, and other oligonucleotides that hybridize to at least a portion of the target nucleic acid. As such, these oligonucleotides may be introduced in the form of single-stranded, double-stranded, circular or hairpin oligonucleotides and may contain structural elements such as internal or terminal bulges or loops. Once introduced to a system, the compositions of the invention may elicit the action of one or more enzymes or structural proteins to effect modification of the target nucleic acid.

One non-limiting example of such an enzyme is RNAse H, a cellular endonuclease which cleaves the RNA strand of an RNA:DNA duplex. Single-stranded antisense oligonucleotides that are “DNA-like” elicit RNAse H. Activation of RNase H, therefore, results in cleavage of the RNA target, thereby greatly enhancing the efficiency of oligonucleotide-mediated inhibition of gene expression. Similar roles have been postulated for other ribonucleases such as those in the RNase III and ribonuclease L family of enzymes.

While a suitable form of antisense oligonucleotide is a single-stranded antisense oligonucleotide, in many species the introduction of double-stranded structures, such as double-stranded RNA (dsRNA) molecules, has been shown to induce potent and specific antisense-mediated reduction of the function of a gene or its associated gene products. This phenomenon occurs in both plants and animals and is believed to have an evolutionary connection to viral defense and transposon silencing.

In the context of this invention, the term “oligonucleoside” refers to a sequence of nucleosides that are joined by internucleoside linkages that do not have phosphorus atoms. Internucleoside linkages of this type include short chain alkyl, cycloalkyl, mixed heteroatom alkyl, mixed heteroatom cycloalkyl, one or more short chain heteroatomic and one or more short chain heterocyclic. These internucleoside linkages include but are not limited to siloxane, sulfide, sulfoxide, sulfone, acetyl, formacetyl, thioformacetyl, methylene formacetyl, thioformacetyl, alkeneyl, sulfamate; methyleneimino, methylenehydrazino, sulfonate, sulfonamide, amide and others having mixed N, O, S, and CH₂ component parts.

Representative United States patents that teach the preparation of the above oligonucleosides include, but are not limited to, U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; 5,792,608; 5,646,269 and 5,677,439, certain of which are commonly owned with this application, and each of which is herein incorporated by reference in its entirety.

In addition to the modifications described above, the nucleosides of the compositions of the invention can have a variety of other modifications so long as these other modifications either alone or in combination with other nucleosides enhance one or more of the desired properties described above. Thus, for nucleotides that are incorporated into compositions of the invention, these nucleotides can have sugar portions that correspond to naturally-occurring sugars or modified sugars. Representative modified sugars include carbocyclic or acyclic sugars, sugars having substituent groups at one or more of their 2′, 3′ or 4′ positions and sugars having substituents in place of one or more hydrogen atoms of the sugar. Additional nucleosides amenable to the present invention having altered base moieties and or altered sugar moieties are disclosed in U.S. Pat. No. 3,687,808 and PCT application PCT/US89/02323.

Oligomeric compounds having altered base moieties or altered sugar moieties are also included in the present invention. All such modified oligomeric compounds are comprehended by this invention so long as they function effectively to mimic the structure of a desired RNA or DNA strand. A class of representative base modifications include tricyclic cytosine analog, termed “G clamp” (Lin et al., J. Am. Chem. Soc., 1998, 120, 8531). This analog makes four hydrogen bonds to a complementary guanine (G) within a helix by simultaneously recognizing the Watson-Crick and Hoogsteen faces of the targeted G. This G clamp modification when incorporated into phosphorothioate oligonucleotides, dramatically enhances antisense potencies in cell culture. The compositions of the invention also can include phenoxazine-substituted bases of the type disclosed by Flanagan et al., Nat. Biotechnol., 1999, 17, 48-52.

The oligomeric compounds in accordance with this invention preferably comprise from about 8 to about 80 monomeric subunits (i.e. from about 8 to about 80 linked nucleosides). One of ordinary skill in the art will appreciate that the invention embodies oligomeric compounds of 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 monomeric subunits in length.

In some embodiments, the oligomeric compounds of the invention are 12 to 50 monomeric subunits in length. One having ordinary skill in the art will appreciate that this embodies oligomeric compounds of 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 monomeric subunits in length.

In other embodiments, the oligomeric compounds of the invention are 15 to 30 monomeric subunits in length. One having ordinary skill in the art will appreciate that this embodies oligomeric compounds of 15, 16, 17, 18, 19, 20, 21, 22, 23, 24, 25, 26, 27, 28, 29, or 30 monomeric subunits in length.

It is not necessary for all positions in an oligomeric compound to be uniformly modified, and in fact more than one of the aforementioned modifications may be incorporated in a single oligomeric compound or even at a single monomeric subunit such as a nucleoside within an oligonucleotide. The present invention also includes chimeric oligomeric compounds such as chimeric oligonucleotides. “Chimeric” oligomeric compounds or “chimeras,” in the context of this invention, are oligomeric compounds such as oligonucleotides containing two or more chemically distinct regions, each made up of at least one monomer unit, i.e., a nucleotide in the case of a nucleic acid based oligomer.

Chimeric oligomeric compounds typically contain at least one region modified so as to confer increased resistance to nuclease degradation, increased cellular uptake, and/or increased binding affinity for the target nucleic acid. An additional region of the oligonucleotide may serve as a substrate for enzymes capable of cleaving RNA:DNA or RNA:RNA hybrids. By way of example, RNase H is a cellular endonuclease which cleaves the RNA strand of an RNA:DNA duplex. Activation of RNase H, therefore, results in cleavage of the RNA target, thereby greatly enhancing the efficiency of inhibition of gene expression. Consequently, comparable results can often be obtained with shorter oligonucleotides when chimeras are used, compared to for example phosphorothioate deoxyoligonucleotides hybridizing to the same target region. Cleavage of the RNA target can be routinely detected by gel electrophoresis and, if necessary, associated nucleic acid hybridization techniques known in the art.

Chimeric compositions of the invention may be formed as composite structures of two or more oligomeric compounds such as oligonucleotides, oligonucleotide analogs, oligonucleosides and/or oligonucleotide mimetics as described above. Such oligomeric compounds have also been referred to in the art as hybrids hemimers, gapmers or inverted gapmers. Representative United States patents that teach the preparation of such hybrid structures include, but are not limited to, U.S. Pat. Nos. 5,013,830; 5,149,797; 5,220,007; 5,256,775; 5,366,878; 5,403,711; 5,491,133; 5,565,350; 5,623,065; 5,652,355; 5,652,356; and 5,700,922, certain of which are commonly owned with the instant application, and each of which is herein incorporated by reference in its entirety.

Specific examples of suitable oligomeric compounds useful in this invention include oligonucleotides containing modified e.g. non-naturally occurring internucleoside linkages. As defined in this specification, oligonucleotides having modified internucleoside linkages include internucleoside linkages that retain a phosphorus atom and internucleoside linkages that do not have a phosphorus atom. For the purposes of this specification, and as sometimes referenced in the art, modified oligonucleotides that do not have a phosphorus atom in their internucleoside backbone can also be considered to be oligonucleosides.

In the C. elegans system, modification of the internucleotide linkage (phosphorothioate) did not significantly interfere with RNAi activity. Based on this observation, it is suggested that certain preferred compositions of the invention can also have one or more modified internucleoside linkages. A suitable phosphorus containing modified internucleoside linkage is the phosphorothioate internucleoside linkage.

Suitable modified oligonucleotide backbones containing a phosphorus atom therein include, for example, phosphorothioates, chiral phosphorothioates, phosphorodithioates, phosphotriesters, aminoalkylphosphotriesters, methyl and other alkyl phosphonates including 3′-alkylene phosphonates, 5′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, selenophosphates and borano-phosphates having normal 3′-5′ linkages, 2′-5′ linked analogs of these, and those having inverted polarity wherein one or more internucleotide linkages is a 3′ to 3′, 5′ to 5′ or 2′ to 2′ linkage. Suitable oligonucleotides having inverted polarity comprise a single 3′ to 3′ linkage at the 3′-most internucleotide linkage, i.e. a single inverted nucleoside residue that may be a basic (the nucleobase is missing or has a hydroxyl group in place thereof). Various salts, mixed salts and free acid forms are also included.

Representative United States patents that teach the preparation of the above phosphorus-containing linkages include, but are not limited to, U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; 5,194,599; 5,565,555; 5,527,899; 5,721,218; 5,672,697 and 5,625,050, certain of which are commonly owned with this application, and each of which is herein incorporated by reference.

In some embodiments of the invention, oligonucleotides have one or more phosphorothioate and/or heteroatom internucleoside linkages, in particular —CH₂—NH—O—CH₂—, —CH₂—N(CH₃)—O—CH₂— (known as a methylene (methylimino) or MMI backbone), —CH₂—O—N(CH₃)—CH₂—, —CH₂—N(CH₃)—N(CH₃)—CH₂— and —O—N(CH₃)—CH₂—CH₂— (wherein the native phosphodiester internucleotide linkage is represented as —O—P(═O)(OH)—O—CH₂—). The MMI type internucleoside linkages are disclosed in the above referenced U.S. Pat. No. 5,489,677. Suitable amide internucleoside linkages are disclosed in the above referenced U.S. Pat. No. 5,602,240.

Suitable modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH₂ component parts.

Representative United States patents that teach the preparation of the above oligonucleosides include, but are not limited to, U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; 5,792,608; 5,646,269 and 5,677,439, certain of which are commonly owned with this application, and each of which is herein incorporated by reference.

In addition to having a 2′-O-methyl modified nucleiside the compositions of the present invention may also contain additional modified sugar moieties. Suitable modified sugar moieties comprise a sugar substituent group selected from: OH; F; O—, S—, or N-alkyl; O—, S—, or N-alkenyl; O—, S— or N-alkynyl; or O-alkyl-O-alkyl, wherein the alkyl, alkenyl and alkynyl may be substituted or unsubstituted C₁ to C₁₀ alkyl or C₂ to C₁₀ alkenyl and alkynyl. Particularly suitable are O((CH₂)ₙO)ₘ—CH₃, O(CH₂)ₙOCH₃, O(CH₂)ₙNH₂, O(CH₂)ₙCH₃, O(CH₂)ₙONH₂, and O(CH₂)ₙON—((CH₂)ₙCH₃)₂, where n and m are from 1 to about 10. Other suitable sugar substituent groups include: C₁ to C₁₀ lower alkyl, substituted lower alkyl, alkenyl, alkynyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, alkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. A suitable modification includes 2′-methoxyethoxy (2′-O—CH₂CH₂OCH₃, also known as 2′-O-(2-methoxyethyl) or 2′-MOE) (Martin et al., Helv. Chim. Acta, 1995, 78, 486-504) i.e., an alkoxyalkoxy group. A further preferred modification includes 2′-dimethylamino-oxyethoxy, i.e., a O(CH₂)₂ON(CH₃)₂ group, also known as 2′-DMAOE, as described in examples hereinbelow, and 2′-dimethylamino-ethoxyethoxy (also known in the art as 2′-O-dimethyl-amino-ethoxy-ethyl or 2′-DMAEOE), i.e., 2′-O—CH₂—O—CH₂—N(CH₃)₂.

Other suitable sugar substituent groups include methoxy (—O—CH₃), aminopropoxy (—OCH₂CH₂CH₂NH₂), allyl (—CH₂—CH═CH₂), —O-allyl (—O—CH₂—CH=CH₂) and fluoro (F). 2′-Sugar substituent groups may be in the arabino (up) position or ribo (down) position. A preferred 2′-arabino modification is 2′-F. Similar modifications may also be made at other positions on the oligomeric compound, particularly the 3′ position of the sugar on the 3′ terminal nucleoside or in 2′-5′ linked oligonucleotides and the 5′ position of 5′ terminal nucleotide. Oligonucleotides may also have sugar mimetics such as cyclobutyl moieties in place of the pentofuranosyl sugar. Representative United States patents that teach the preparation of such modified sugar structures include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; 5,792,747; and 5,700,920, certain of which are commonly owned with the instant application, and each of which is herein incorporated by reference in its entirety.

Particularly suitable sugar substituent groups include O((CH₂)ₙO)ₘCH₃, O(CH₂)ₙOCH₃, O(CH₂)ₙNH₂, O(CH₂)ₙCH₃, O(CH₂)ₙONH₂, and O(CH₂)ₙON((CH₂)ₙCH₃))₂, where n and m are from 1 to about 10.

Oligomeric compounds including oligonucleotides may also include nucleobase (often referred to in the art simply as “base” or “heterocyclic base moiety”) modifications or substitutions. As used herein, “unmodified” or “natural” nucleobases include the purine bases adenine (A) and guanine (G), and the pyrimidine bases thymine (T), cytosine (C) and uracil (U). Modified nucleobases also referred herein as heterocyclic base moieties include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl (—C≡C—CH₃) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyl-adenine, 2-F-adenine, 2-amino-adenine, 8-azaguanine and 8-azaadenine, 7-deaza-guanine and 7-deazaadenine and 3-deazaguanine and 3-deazaadenine.

Heterocyclic base moieties may also include those in which the purine or pyrimidine base is replaced with other heterocycles, for example 7-deaza-adenine, 7-deazaguanosine, 2-aminopyridine and 2-pyridone. Further nucleobases include those disclosed in U.S. Pat. No. 3,687,808, those disclosed in The Concise Encyclopedia Of Polymer Science And Engineering, pages 858-859, Kroschwitz, J. I., ed. John Wiley & Sons, 1990, those disclosed by Englisch et al., Angewandte Chemie, International Edition, 1991, 30, 613, and those disclosed by Sanghvi, Y. S., Chapter 15, Antisense Research and Applications, pages 289-302, Crooke, S. T. and Lebleu, B. , ed., CRC Press, 1993. Certain of these nucleobases are particularly useful for increasing the binding affinity of the compositions of the invention. These include 5-substituted pyrimidines, 6-azapyrimidines and N-2, N-6 and O-6 substituted purines, including 2 aminopropyladenine, 5-propynyluracil and 5-propynylcytosine. 5 methylcytosine substitutions have been shown to increase nucleic acid duplex stability by 0.6-1.2° C. (Sanghvi, Y. S., Crooke, S. T. and Lebleu, B., eds., Antisense Research and Applications, CRC Press, Boca Raton, 1993, pp. 276-278) and are presently preferred base substitutions, even more particularly when combined with 2′-O-methoxyethyl sugar modifications.

As used herein, “bioactivity in vivo” is any activity within a cell in vivo including, but not limited to, alteration of the level of an RNA molecule to which the oligomeric compound is targeted, or alteration of a protein encoded by an RNA molecule to which the oligomeric compound is targeted.

As used herein, “a bioindicative cell” is any cell in which an in vitro activity of an oligomeric compound is observed and is correlated to an in vivo activity of the same oligomeric compound. Bioindicative cells include, but are not limited to, mammalian tissue-derived cells such as, for example, primary hepatocytes, primary keratinocytes, primary macrophages, primary fibroblasts, primary pancreatic cells, or stem cells.

As used herein, “altered phenotype” is any phenotypic trait for which an alteration can be observed. Altered phenotypes include, but are not limited to, an increase in uptake of the candidate oligomeric compound, decrease in expression of the RNA produced from the gene to which the candidate oligomeric compound is targeted, or decrease in expression of the protein encoded by the gene to which the candidate oligomeric compound is targeted.

As used herein, “transfection reagent” is any reagent that enhances transfection of an oligomeric compound into a cell. Transfection reagents are well known to the skilled artisan.

As used herein, “assay platform” is any platform in which a cell-based assay can be carried out including, but not limited to, a 96-well microtiter plate, a 48-well microtiter plate, a 6-well microtiter plate, and the like.

The present invention provides methods of identifying an oligomeric compound having bioactivity in vivo. A bioindicative cell is contacted with one or more candidate oligomeric compounds in vitro in the absence of a transfection reagent. Contacting can occur by any means known to those skilled in the art. The bioindicative cell is examined to determine whether it has an altered phenotype. Such examination can be carried out via morphological analysis, biochemical analysis, or the like. If the bioindicative cell has an altered phenotype, one or more of the candidate oligomeric compounds comprises in vivo bioactivity.

In some embodiments, the oligomeric compound is single stranded. Alternately, the oligomeric compound can be double stranded. In some embodiments, the oligomeric compound is an oligonucleotide, peptide nucleic acid, small interfering RNA, micro RNA, micro RNA mimic, or any combination thereof.

In some embodiments, the oligomeric compound is chemically modified. In other embodiments, the oligomeric compound is a gapmer. In some embodiments, the gapmer comprises two 2′-O-methoxyethyl, 2′-O-methyl, 2′-methyl, or 2′-F wings. In some embodiments, the oligomeric compound comprises phosphorothioate internucleoside linkages.

In some embodiments, the bioindicative cell is a mammalian tissue-derived cell including, but not limited to, a primary hepatocyte, primary keratinocyte, primary macrophage, primary fibroblast, primary pancreatic cell, or a stem cell. In some embodiments, the mammalian tissue-derived cell is a rodent (i.e., mouse or rat) primary hepatocyte. In other embodiments, the mammalian tissue-derived cell is a primate primary hepatocyte. Primates include, but are not limited to, monkeys (i.e., Cynomolgus) and humans.

Altered phenotypes include, but are not limited to, an increase in uptake of the candidate oligomeric compound, decrease in expression of the mRNA produced from the gene to which the candidate oligomeric compound is targeted, or decrease in expression of the protein encoded by the gene or mRNA to which the candidate oligomeric compound is targeted.

In some embodiments, the candidate oligomeric compound is designed to inhibit gene expression by hybridizing to a target through an antisense mechanism such as, for example, an RNAse H-mediated inhibition of the target of the candidate oligomeric compound, an RNA interference-mediated inhibition of the target of the candidate oligomeric compound, or splicing.

In other embodiments, the candidate oligomeric compound is designed to inhibit RNA metabolism, transport, or protein metabolism by hybridizing to a target through an antisense mechanism.

The present invention also provides kits comprising an assay platform, a bioindicative cell, and a bioactive oligomeric compound.

The present invention also provides methods of identifying an oligomeric compound having bioactivity in vivo in which a primary hepatocyte is contacted with a candidate oligomeric compound in vitro in the absence of a transfection reagent. The primary hepatocyte is examined to determine whether it has a decreased level of an RNA to which the candidate oligomeric compound is targeted. If the primary hepatocyte has a decreased level of the RNA, then the candidate oligomeric compound comprises in vivo bioactivity.

The present invention also provides methods of identifying a small interfering RNA having bioactivity in vivo. A primary hepatocyte is contacted with a candidate small interfering RNA in vitro in the absence of a transfection reagent. The primary hepatocyte is examined to determine whether it has a decreased level of an RNA to which the candidate small interfering RNA is targeted. If the primary hepatocyte has a decreased level of the RNA, then the candidate small interfering RNA comprises in vivo bioactivity.

In order that the invention disclosed herein may be more efficiently understood, examples are provided below. It should be understood that these examples are for illustrative purposes only and are not to be construed as limiting the invention in any manner. Throughout these examples, molecular cloning reactions, and other standard recombinant DNA techniques, were carried out according to methods described in Maniatis et al., Molecular Cloning—A Laboratory Manual, 2nd ed., Cold Spring Harbor Press (1989), using commercially available reagents, except where otherwise noted.

### EXAMPLES

### Example 1

### Animals

Balb/c mice, 18-24 g (5-7 weeks old), were obtained from Charles River (Wilmington Mass.) and used for subsequent in vitro and in vivo experiments. Animals were housed in polycarbonate cages and given access to chow and water ad libitum, in accordance with protocols approved by the Institutional Animal Care and Use Committee.

### Example 2

### Oligonucleotides

All chimeric MOE Gapmers are 20-mer phosphorothioate oligodeoxynucleotides containing 2′-O-methoxyethyl (2′-MOE) modifications at positions 1-5 and 16-20. MOE Gapmers and the 2′-deoxy unmodified phosphorothioate oligodeoxynucleotides ODN-PTEN and ODN-PTEN(6MM) were synthesized on a Milligen model 8800 DNA synthesizer (Millipore Inc., Bedford Mass.) using conventional solid-phase triester chemistry (Sanghvi, 1999) at Isis Pharmaceuticals Inc. Deprotected and desalted siRNA analogs were obtained from Dharmacon Research, Inc. (LaFayette, Colo.). Sequences of siRNA compounds, and the oligonucleotides and placement of their 2′-O-methoxyethyl modifications, are detailed in Table 1. 2′-MOE Gapmers (MG) are first generation 20-mer phosphorothioate oligodeoxynucleotides which contain 2′-O-methoxyethyl (2′-MOE) modifications at positions 1-5 and 16-20 (boldface type). ISIS 160847 and 160848 are first generation phosphorothioate oligodeoxynucleotides (ODN). Both antisense and sense strands are shown for each siRNA construct (si). Six-base mismatch (6MM) control oligonucleotides are of similar nucleoside composition as the respective antisense oligonucleotides.

TABLE 1

| | | | | Sequence | SEQ ID | |
|---|---|---|---|---|---|---|
| | Isis No. | Target | Strand | (5′ to 3′) | NO: | Composition |
| ASOs | | | | | | |
| | 160847 | PTEN | as | CTGCTAGCCTCTGGATTTGA | 16 | 2′-deoxy P = S; 5- |
| | | | | | | MeC |
| ODN- | 160848 | PTEN | as | CTTCTGGCATCCGGTTTAGA | 17 | 2′-deoxy P = S; 5- |
| PTEN | | | (6MM) | | | MeC |
| (6MM) | | | | | | |
| MG-PTEN | 116847 | PTEN | as | CTGCTAGCCTCTGGATTTGA | 18 | 5_10_5 2′MOE |
| | | | | | | Gapmer; 5-MeC |
| MG-PTEN | 116848 | PTEN | as | CTTCTGGCATCCGGTTTAGA | 19 | 5_10_5 2′MOE |
| (6MM) | | | (6MM) | | | Gapmer; 5-MeC |
| MG-Fas | 22023 | Fas | as | TCCAGCACTTTCTTTTCCGG | 20 | 5_10_5 2′MOE |
| | | | | | | Gapmer; 5-MeC |
| MG-Fas | 29836 | Fas | as | TCCATCTCCTTTTATGCCGG | 21 | 5_10_5 2′MOE |
| (6MM) | | | (6MM) | | | Gapmer; 5-MeC |
| MG-MTTP | 144477 | MTTP | as | CCCAGCACCTGGTTTGCCGT | 22 | 5_10_5 2′MOE |
| | | | | | | Gapmer; 5-MeC |
| MG-ApoB | 147764 | Apo B | as | GTCCCTGAAGATGTCAATGC | 23 | 5_10_5 2′MOE |
| | | | | | | Gapmer; 5-MeC |
| siRNAs† | | | | | | |
| si-PTEN | 263186 | PTEN | as | CU*GC*UA*GC*CU*CU*GG*AU*UU*GdT*dT | 24 | alt *P = S, P = O |
| | | | | | | linkage; 3′-dTdT |
| | | | | | | overhang |
| | 263187 | PTEN | s | CA*AA*UC*CA*GA*GG*CU*AG*CA*GdT*dT | 25 | alt *P = S, P = O |
| | | | | | | linkage; 3′-dTdT |
| | | | | | | overhang |
| si-PTEN | 263188 | PTEN | as | CU*UC*UG*GC*AU*CC*GG*UU*UA*GdT*dT | 26 | alt *P = S, P = O |
| (6MM) | | | (6MM) | | | linkage; 3′-dTdT |
| | | | | | | overhang |
| | 263189 | PTEN | s | CU*AA*AC*CG*GA*UG*CC*AG*AA*GdT*dT | 27 | alt *P = S, P = O |
| | | | (6MM) | | | linkage; 3′-dTdT |
| | | | | | | overhang |
| si-PTEN | 278626 | PTEN | as | CUGCUAGCCUCUGGAUUUGAC | 28 | unmodified RNA |
| (blunt) | | | | | | |
| | 278627 | PTEN | s | GUCAAAUCCAGAGGCUAGCAG | 29 | unmodified RNA |
| si-Fas‡ | — | Fas | as | 5′-P | 30 | unmodified RNA; |
| | | | | GUCUGGUUUGCACUUGCACdTdT | | 5′-Phosphate, 3′- |
| | | | | | | dTdT overhang |
| | — | Fas | s | 5′-P | 31 | unmodified RNA; |
| | | | | GUGCAAGUGCAAACCAGACdTdT | | 5′-Phosphate, 3′- |
| | | | | | | dTdT overhang |
| si-Fas | 328798 | Fas | as | 5′-P | 32 | unmodified RNA; |
| (6MM) | | | (6MM) | GUGUCGUGUUCAGUUCCACdTdT | | 5′-Phosphate, 3′- |
| | | | | | | dTdT overhang |
| | 328799 | Fas | s | 5′-P | 33 | unmodified RNA; |
| | | | (6MM) | GUGGAACUGAACACGACACdTdT | | 5′-Phosphate, 3′- |
| | | | | | | dTdT overhang |

† siRNAs are named as dsRNA sets (e.g. si-PTEN includes the antisense strand 263186 and sense strand 263187)
‡ si-Fas sequences from Song et. al. (2003)
Legend:
as—antisense strand,
s—sense strand,
ApoB—Apolipoprotein B,
PTEN—Phosphotase and Tensin homolog deleted on chromosome Ten,
MTTP—Microsomal Triglyceride Transfer Protein

### Example 3

### In Vitro Analysis

Primary hepatocyte isolation/culture. Mouse hepatocytes were isolated from mice using a two step in situ liver perfusion as previously described (McQueen et al., Cell. Biol. Toxicol., 1989, 5, 201-206). Briefly, animals were anesthetized with Avertin (50 mg/kg, intraperitoneal) and the portal vein was exposed. Hank's Balanced Salt Solution (Life Technologies, Grand Island, N.Y.) was perfused through the portal vein for 3.5 min at 2 ml/min followed by Williams Medium E (WME: Life Technologies, Grand Island, N.Y.) containing 0.3 mg/ml collagenase B (Roche Molecular Biochemicals, Indianapolis, Ind.) for 5.5 minutes. The liver was removed from the animal and gently massaged through Nitex nylon mesh (Tetko, Depew, N.Y.) to obtain a suspension of cells. The suspension was centrifuged (4 minutes at 500 rpm) and the supernatant discarded. The remaining pellet was gently resuspended in WME and centrifuged (4 minutes at 500 rpm) two more times to remove nonparenchymal cells. The pelleted hepatocytes were resuspended in WME supplemented with 10% fetal bovine serum (FBS)(v:v) and the concentration of cells was determined. For plating, cells were resuspended to the desired working concentration in WME supplemented with 10% FBS, 1% L-glutamine (v:v), 1% HEPES (v:v), and 1% gentamycin (antimitotic-antibiotic). Cells were plated on Primaria™ coated 6-well plates at a density of 100,000 per ml or Primaria™ 96-well coated plates at a density of 10,000 per ml. Cells were allowed to adhere to plates for one hour and then gently washed with PBS to remove dead cells and the media replaced with fresh HepatoZYME™ media (Invtirogen, Carlsbad, Calif.) supplemented with 1% L-glutamine (v:v), 1% HEPES (v:v), 1% non-essential amino acids (NEAA) and 1% gentamycin (antimitotic-antibiotic).

In vitro hepatocyte oligodeoxynucleotide transfections. For experiments transfecting primary hepatocytes with cationic lipids, transfections were performed either four hours after plating or after an additional 8-12 hours (overnight). No difference in transfection results were observed comparing the two plating intervals (data not shown). The oligonucleotide or siRNA (oligo/siRNA) was mixed with Lipofectin (Invitrogen, Carlsbad, Calif.) at a working concentration of 3 μg per 100 nM of single strand DNA or RNA per 1 ml of media. Prior to addition to cells, the mix was incubated 5-10 minutes as per vendor recommendations. Plating media was then removed and the cells were treated for 4-6 hours, the media changed to fresh HepatoZYMETM (supplemented as above) and cells incubated overnight for an additional 16-20 hours. Cells were then lysed and the RNA isolated and purified as described below. For free uptake studies, cells were allowed to adhere to the plastic for 4 hours then treated with the oligos/siRNA in the HepatoZYMETM media for 12-16 hours (overnight). Cells were then lysed and the RNA isolated and purified as described below.

RNA isolation and expression analysis. In vitro total RNA was harvested at the indicated times post-transfection using the RNeasy Mini kit (Qiagen, Valencia, Calif.) for the 6-well treatments and using the Qiagen BioRobot 3000 for the 96-well plates, according to the manufacturers protocol. Gene expression was determined via real time quantitative RT-PCR on the ABI Prism 7700 system (Applied Biosystems, Foster City Calif.) as suggested by the manufacturer and described in the literature (Gibson et al., Genome Res., 1996, 6, 995-1001; Winer et al., Anal. Biochem., 1999, 270, 41-49; and Vickers et al., J. Biol. Chem., 2003, 278, 7108-7118). Primers and probes were obtained from IDT, Inc. (Coralville, Iowa) and the following primer/probe sets were used: PTEN (accession number U92437.1), forward primer (ATGACAATCATGTTGCAGCAATTC; SEQ ID NO:1), reverse primer (CGATGCAATAAATATGCACAAATCA; SEQ ID NO:2), and probe (GTAAAGCTGGAAAGGGACGGACTGGT; SEQ ID NO:3); Fas (accession number M83649.1), forward primer (TCCAAGACACAGCTGAGCAGA; SEQ ID NO:4), reverse primer (TGCATCACTCTTCCCATGAGAT; SEQ ID NO:5), and probe (AGTCCAGCTGCTCCTGTGCTGGTACC; SEQ ID NO:6); Apolipoprotein B (accession number M35186.1), forward primer (CGTGGGCTCCAGCATTCTA; SEQ ID NO:7), reverse primer (AGTCATTTCTGCCTTTGCGTC; SEQ ID NO:8), and probe (CCAATGGTCGGGCACTGCTCAA; SEQ ID NO:9); Microsomal triglyceride transfer protein (accession number NM_{—}008642.1), forward primer (GAGCGGTCTGGATTTACAACG; SEQ ID NO:10), reverse primer (AGGTAGTGACAGATGTGGCTTTTG; SEQ ID NO:11), and probe (CAAACCAGGTGCTGGGCGTCAGT; SEQ ID NO:12); and murine cyclophilin A (accession number), forward primer (TCGCCGCTTGCTGCA; SEQ ID NO:13), reverse primer (ATCGGCCGTGATGTCGA; SEQ ID NO:14) and probe (CCATGGTCAACCCCACCGTGTTC; SEQ ID NO:15). Cyclophilin A mRNA levels were used with 96-weel transfection experiments as an internal standard for sample to sample normalization. All mRNA expression levels were normalized both to RiboGreen (Molecular Probes, Eugene, Oreg.), and GAPDH mRNA, also determined by quantitave RT-PCR (data not shown), from the same total RNA samples. Dose-response trends were independent of the normalization technique, and only RiboGreen normalized data is presented here.

Statistical Analysis. Simple Student's T-Test were performed.

Primary hepatocyte monolayer model. Mouse primary hepatocytes plated in 6-well plates were dosed with ISIS 116847(MG-PTEN), a MOE gapmer specific for PTEN (Butler et al., Diabetes, 2002, 51, 1028-1034), at 25 and 100 nM in the presence of lipofectin. PTEN mRNA expression levels fell in a dose-dependent manner. Transcript expression was reduced by a maximum of 87% (0.13±0.06 of control) at 100 nM, with an IC50 of approximately 25 nM. Doses above 100 nM did not significantly decrease message knockdown (data not shown).

### Example 4

### In Vivo Analysis

In vivo oligonucleotide treatment. MOE gapmer or siRNA oligonucleotides were administered in saline (0.9% NaCl) via intravenous tail vein injection at the indicated doses once per day for five days. Mice were sacrificed on day five, six hours post administration. Liver RNA was isolated as described below.

RNA isolation and expression analysis. Total RNA was extracted from mouse liver by homogenizing liver in guanidinium isothiocyanate at time of sacrifice, and isolating total RNA standard cesium chloride gradient centrifugation techniques. Gene expression was determined via real time quantitative RT-PCR on the ABI Prism 7700 system (Applied Biosystems, Foster City Calif.) as described above.

Primary hepatocyte monolayer model. Mouse primary hepatocytes plated in 6-well plates were dosed with ISIS 116847(MG-PTEN), a MOE gapmer specific for PTEN (Butler et al., Diabetes, 2002, 51, 1028-1034), at 25 and 100 nM in the presence of lipofectin PTEN mRNA expression levels fell in a dose-dependent manner. Transcript expression was reduced by a maximum of 87% (0.13±0.06 of control) at 100 nM, with an IC50 of approximately 25 nM. Doses above 100 nM did not significantly decrease message knockdown (data not shown).

### Example 5

### Design of Single and Double-Strand Antisense Constructs

MG-PTEN is a 20-base chimeric 2′-O-methoxyethyl oligonucleotide (MOE gapmer) that has previously been demonstrated to be a potent inhibitor of mouse PTEN expression (Butler et al., Diabetes, 2002, 51, 1028-1034). siRNA analogs to the same coding region targeted by MG-PTEN were synthesized to compare the in vitro dose-response characteristics of the two classes of antisense compounds. Table 1 is a complete list of oligonucleotides used, their sequences, and specific chemical modifications.

Single-strand MOE gapmers and double-strand RNA (dsRNA) show comparable activity profiles in primary mouse hepatocytes under cationic lipid transfection conditions.

Mouse primary hepatocytes plated in 96-well plates were transfected with either: the MOE gapmer MG-PTEN, the 6-base mismatch (MG-PTEN(6MM)), the blunt ended dsRNA analog to the MOE 116847 site (si-PTEN(blunt)), the 2-nt 3′-overhang dsRNA analog with mixed backbone (si-PTEN), or the 6 base-pair 2-nt 3′-overhang dsRNA mismatch to si-PTEN with mixed backbone (si-PTEN(6MM)) in the presence of Lipofectin. Both the MOE gapmer and the dsRNA designed against the target region 116847 significantly reduced PTEN mRNA in a dose-dependent manner. The mixed backbone dsRNA containing 2-nt 3′-dTdT overhangs, si-PTEN, appeared to have a slightly lower IC50 than the corresponding blunt-end dsRNA, si-PTEN (blunt). While, the IC50 for the 2′-MOE MG-PTEN was significantly lower (12.5 nM), maximal mRNA reduction was achieved at 200 nM (higher dosages not shown). The lower IC50 observed for MG-PTEN could reflect mechanistic differences in target reduction or reflect that the sequence used for comparative purposes was optimized for MOE gapmer chemistry. The PTEN mismatch control to the mixed backbone dsRNA containing 2-nt 3′-dTdT overhangs, si-PTEN(6MM), did not effect PTEN mRNA levels, suggesting that target reduction was not due to non-specific dsRNA or siRNA effects.

Uptake activity is independent of sequence. Evidence suggests that the uptake of antisense oligonucleotides is independent of oligonucleotide sequence (Leeds et al., Nucleosides Nucleotides, 1997, 16, 1689-1693; and Geary et al., J. Pharmacol. Exp., 2001, 296, 890-897). To confirm that the in vitro Lipofectin mediated dose-dependent inhibition of target observed with the MOE gapmer MG-PTEN could be reproduced with other potent antisense MOE gapmers, another potent MOE gapmer, MG-Fas, was selected for Lipofectin mediated dose-response analysis. MG-Fas targets a sequence within the translated region of the murine Fas transcript. It is a 20-base chimeric MOE gapmer that has been shown to inhibit Fas expression both in vitro and in vivo in a dose-dependent and sequence-specific manner. It has been reported that both Fas mRNA and protein levels fall as much as 90% in mice dosed with MG-Fas (Zhang et al., Nat. Biotechnol., 2000, 18, 862-867). As described for PTEN, mouse primary hepatocytes were plated in 96-well plates and transfected with either: the MOE gapmer MG-Fas; MG-Fas(6MM), a 6 base mismatch control to MG-Fas; si-Fas, a dsRNA containing an antisense strand using the anti-Fas siRNA sequence 1 from a study by Song et. al. (Nat. Med., 2003, 9, 347-351), where they reported that hydrodynamic tail vein injection of this sequence into mice reduced Fas mRNA expression in liver hepatocytes by approximately 86% of control; and si-Fas(6MM), a 6 base mismatch control dsRNA. Both MG-Fas and si-Fas reduced Fas mRNA expression in a dose-dependent manner, MG-Fas reducing Fas mRNA to 0.76±0.12 and 0.04±0.03 of control at 75 and 300 nM, respectively; and si-Fas reducing expression to 0.82±0.05 and 0.29±0.08 of control at 75 and 300 nM, respectively. Thus, the PTEN and Fas data taken together suggest that both MOE gapmers and dsRNAs inhibit gene expression in a dose-dependent manner when transfected into isolated mouse hepatocytes.

To further investigate whether chemical modifications to the MOE gapmer backbone would alter dose-response characteristics, mouse primary hepatocytes were transfected with either the MOE gapmer MG-PTEN; MG-PTEN(6MM), the 6 base mismatch control to MG-PTEN; ODN-PTEN, a first generation unmodified 20-mer phosphorothioate (PS) oligonucleotide (no MOE modifications); or ODN-PTEN(6MM), ODN-PTEN's 6 base mismatch control (Table 1). These oligodeoxynucleotides contain PS backbones, but are uniformly 2′-OH unmodified. Both the MOE gapmer and the unmodified PS oligonucleotide significantly reduced PTEN mRNA in a dose-sensitive manner. The slightly greater target knockdown seen with the MG-PTEN supports previous observations that MOE modified PS oligonucleotides have slightly increased binding affinity for their complementary RNAs (Crooke et al., Biochem. J., 1995, 312(Pt 2), 599-608). MOE gapmer increased nuclease resistance may also be increasing MG-PTENs efficacy in this assay by increasing intracellular concentrations relative to ODN-PTEN over time.

Single-strand MOE gapmers and PS oligonucleotides show different activity profiles than double-strand RNA (dsRNA) in primary mouse hepatocytes in free-uptake conditions. Graham et al. (J. Pharmacol. Exp. Therap., 1998, 286, 447-458) has previously demonstrated both free uptake and activity of MOE gapmers incubated with primary hepatocytes without the use of cationic lipids similar to that seen in vivo. To investigate the dose-response sensitivity of the MOE gapmers, experiments were conducted in mouse primary hepatocytes plated in 6-well plates with both MG-PTEN and MG-Fas at concentrations ranging from 75 to 10000 nM (see above procedures). The expression levels of both targeted PTEN and Fas mRNA subsequently fell in a dose-dependent manner. Maximal inhibition (˜90%) of both PTEN and Fas mRNA levels was achieved at 3000 nM, with an IC50 of approximately 350 nM for PTEN and 750 nM for Fas. Higher concentrations of either MG-PTEN or MG-Fas did not significantly reduce transcript levels. Six base mismatches to both MG-PTEN and MG-Fas, MG-PTEN(6MM) and MG-Fas(6MM), did not reduce transcript levels; arguing against ASO dose related mRNA toxicity (data not shown). PTEN and Fas transcript levels were normalized with RiboGreen. However, the dose-response trends observed were independent of the normalization technique, as normalization using either RiboGreen or GAPDH transcript levels yielded similar results (GAPDH data not shown).

To further confirm that the in vitro dose-dependent inhibition of target observed with both MG-PTEN and MG-Fas could be reproduced with other potent antisense MOE gapmers, two additional potent MOE gapmers, MG-ApoB and MG-MTTP were selected for free uptake dose-response analysis (see Table 1). MG-ApoB is a potent inhibitor of the mouse apolipoprotein B (ApoB) (unpublished data). MG-MTTP targets mouse microsomal triglyceride transfer protein (MTTP) (unpublished data). All MOE gapmers tested displayed similar dose-response dynamics (data not shown), suggesting that the mechanism of MOE gapmer uptake and subsequent RNA inhibition is highly conserved.

To determine whether dsRNA might also demonstrate both free uptake and activity without the use of cationic lipids, mouse primary hepatocytes were dosed with either si-PTEN(blunt), si-PTEN, si-PTEN(6MM), the MG-PTEN, MG-Fas, MG-Fas(6MM), si-Fas, or si-Fas(6MM); at concentrations ranging from 375 to 1500 nM. The siRNA constructs are capable of specific target reduction in the presence of the cationic lipid Lipofectin. However, whereas the MOE modified single-strand DNAs MG-PTEN and MG-Fas show robust target reduction even at the lowest concentrations (375 nM), no target reduction was observed with dsRNA, suggesting that either: 1) dsRNA are not transported across the cell plasma membrane, 2) dsRNA is transported directly into the nucleus, where it is not accessible to the cytoplasmic RISC complex machinery, or 3) the duplex siRNA is not stable in the media and is either falling apart or being degraded.

Given the lack of activity observed for dsRNA in free uptake experiments, it was of interest to determine whether MOE modifications were aiding free uptake of single-strand DNA. Again, the unmodified homologs to MG-PTEN and the 6 base mismatch MG-PTEN(6MM), ODN-PTEN and ODN-PTEN(6MM) were used. These first generation, unmodified molecules demonstrate a dose responsive, specific target reduction. Again, the MOE modified gapmer MG-PTEN demonstrated much greater message knockout, suggesting that the MOE modification may assist and improve the oligonucleotide delivery in the absence of transfection reagents. Again, the half-lives of unmodified first generation oligodeoxynucleotides are much shorter, which may in part explain the reduced activity observed with these molecules.

Capillary gel electrophoresis (CGE) was used to look at the stability of the duplex siRNA constructs in the treatment media (see above for media description) at different time points. If the duplex is still intact after 16 hrs, which is the duration of our treatments, the construct is considered valid for the in vitro assay proposed herein, and tested for uptake and/or activity.

### Example 6

### In vivo Target Inhibition—MOE Gapmers Versus dsRNAs

Given the observed robust target inhibition with both single-stranded MOE gapmers, unmodified PS oligonucleotides and dsRNA when using Lipofectin as a transfection agent, but no observation of dsRNA activity when a transfection reagent was not used, coupled with reports that dsRNA when administered in vivo via high pressure tail injections knock down target (McCafferey et al., Nature, 2002, 418, 38-39; Lewis et al., Nat. Genet., 2002, 32, 107-108; and Song et al., Nat. Med., 2003, 3, 347-351), it was of interest to compare MOE gapmer and dsRNA activity in vivo using conventional intravenous injections. MG-PTEN, MG-PTEN(6MM), si-PTEN and si-PTEN(6MM) were administered daily for 5 days at concentrations of either 2.5 mg/kg or 25 mg/kg. Only MG-PTEN reduced PTEN mRNA levels in liver. Further, in a separate study, animals were dosed daily for five days with si-PTEN(blunt) to concentrations as high as 50 mg/kg. Again, only MG-PTEN reduced PTEN mRNA levels in liver. No effect was observed for intraperitoneal injected siPTEN(blunt). High-pressure delivery systems may mimic in vitro transfection mediated oligonucleotide delivery by altering cell membrane permeability; however, we are unaware of any studies demonstrating mRNA knockdown with dsRNA using conventional delivery systems.

The results suggest that the in vitro primary hepatocyte model correlates both with single-strand DNA oligonucleotide (both MOE gapmer and PS oligonucleotides) and dsRNA in vivo activity. Specifically, whereas single-strand oligonucleotides effectively decrease target mRNA expression both in vitro and in vivo without the aid of a delivery system, dsRNA does not decrease target mRNA expression in hepatocytes in vitro without the aid of transfection reagents or in vivo when delivered by conventional dosing methods.

Various modifications of the invention, in addition to those described herein, will be apparent to those skilled in the art from the foregoing description. Such modifications are also intended to fall within the scope of the appended claims. Each reference cited in the present application is incorporated herein by reference in its entirety.

1. A method of identifying an oligomeric compound having bioactivity in vivo comprising the steps of:
contacting a bioindicative cell with one or more candidate oligomeric compounds in vitro in the absence of a transfection reagent; and
determining whether the bioindicative cell has an altered phenotype, wherein if the bioindicative cell has an altered phenotype, one or more of the candidate oligomeric compounds comprises in vivo bioactivity.

2. A method of claim 1 wherein the oligomeric compound is single stranded.

3. A method of claim 1 wherein the oligomeric compound is double stranded.

4. A method of claim 1 wherein the oligomeric compound is an oligonucleotide, peptide nucleic acid, small interfering RNA, micro RNA, micro RNA mimic, or any combination thereof.

5. A method of claim 1 wherein the oligomeric compound is chemically modified.

6. A method of claim 5 wherein the oligomeric compound is a gapmer.

7. A method of claim 6 wherein the gapmer comprises two 2′-O-methoxyethyl, 2′-O-methyl, 2′-methyl, or 2′-F wings.

8. A method of claim 1 wherein the oligomeric compound comprises phosphorothioate internucleoside linkages.

9. A method of claim 1 wherein the bioindicative cell is a mammalian tissue-derived cell.

10. A method of claim 9 wherein the mammalian tissue-derived cell is a primary hepatocyte, primary keratinocyte, primary macrophage, primary fibroblast, primary pancreatic cell, or a stem cell.

11. A method of claim 9 wherein the mammalian tissue-derived cell is a rodent primary hepatocyte.

12. A method of claim 11 wherein the rodent is a mouse.

13. A method of claim 11 wherein the rodent is a rat.

14. A method of claim 9 wherein the mammalian tissue-derived cell is a primate primary hepatocyte.

15. A method of claim 14 wherein the primate is a Cynomolgus monkey.

16. A method of claim 14 wherein the primate is a human.

17. A method of claim 11 wherein the altered phenotype is an increase in uptake of the candidate oligomeric compound, decrease in expression of the mRNA produced from the gene to which the candidate oligomeric compound is targeted, or decrease in expression of the protein encoded by the gene or mRNA to which the candidate oligomeric compound is targeted.

18. A method of claim 1 wherein the candidate oligomeric compound is designed to inhibit gene expression by hybridizing to a target through an antisense mechanism.

19. A method of claim 18 wherein the antisense mechanism is an RNAse H-mediated inhibition of the target of the candidate oligomeric compound.

20. A method of claim 18 wherein the antisense mechanism is an RNA interference-mediated inhibition of the target of the candidate oligomeric compound.

21. A method of claim 18 wherein the antisense mechanism is splicing.

22. A method of claim 1 wherein the candidate oligomeric compound is designed to inhibit RNA metabolism by hybridizing to a target through an antisense mechanism.

23. A method of claim 1 wherein the candidate oligomeric compound is designed to inhibit transport by hybridizing to a target through an antisense mechanism.

24. A method of claim 1 wherein the candidate oligomeric compound is designed to inhibit protein metabolism by hybridizing to a target through an antisense mechanism.

25. A kit comprising an assay platform, a bioindicative cell, and a bioactive oligomeric compound.

26. A method of identifying an oligomeric compound having bioactivity in vivo comprising the steps of:
contacting a primary hepatocyte with a candidate oligomeric compound in vitro in the absence of a transfection reagent; and
determining whether the primary hepatocyte has a decreased level of an RNA to which the candidate oligomeric compound is targeted, wherein if the primary hepatocyte has a decreased level of the RNA, then the candidate oligomeric compound comprises in vivo bioactivity.

27. A method of identifying a small interfering RNA having bioactivity in vivo comprising the steps of:
contacting a primary hepatocyte with a candidate small interfering RNA in vitro in the absence of a transfection reagent; and
determining whether the primary hepatocyte has a decreased level of an RNA to which the candidate small interfering RNA is targeted, wherein if the primary hepatocyte has a decreased level of the RNA, then the candidate small interfering RNA comprises in vivo bioactivity.
